(19) United States
(12) Patent Application Publication
(10) Pub. No.: US 2011/0282452 A1
(43) Pub. Date: Nov. 17, 2011

(54) AUXETIC MATERIAL

(75) Inventors: Carolin Koerner, Feucht (DE); Peter Heinl, Nuernberg (DE); Robert Friedrich Singer, Erlangen (DE)

(73) Assignee: Friedrich-Alexander-Universitat Erlangen-Numberg, Erlangen (DE)

(21) Appl. No.: 12/998,610

(22) PCT Filed: Oct. 14, 2009
(86) PCT No.: PCT/EP2009/063414

§ 371 Date: Jul. 29, 2011

(30) Foreign Application Priority Data
Nov. 10, 2008 (DE) 102008043623.2

(51) Int. Cl. A61F 2/28; B32B 1/00

(52) U.S. Cl. 623/16.11; 428/105; 428/603

(57) ABSTRACT

The invention relates to an auxetic material that is composed of a periodic arrangement of three-dimensional structural elements (G, G1, G2, G3) connected to each other, wherein each of the structural elements (G) comprises a first (3) and at least three second supporting elements (4), wherein the first (3) and the second supporting elements (4) are connected at a common node (1) with their one ends, and wherein a first angle (α) between the first supporting element (3) and the second supporting elements (4) is less than 90°.

The invention relates to an auxetic material.

An auxetic material is understood to mean a material with a negative Poisson's ratio ν. Auxetic materials behave abnormally in contrast to materials with a positive Poisson's ratio ν. In other words, when under pressure, they contract in a direction vertical to the direction of pressure, whereas during pulling, they expand in a direction perpendicular to the direction of pulling.

Auxetic materials made from a compressed polymer foam are known from prior art. For example, reference is made to the U.S. Pat. No. 4,668,557, WO 99/25530, U.S. Pat. No. 5,035,713 as well as the WO 2007/052054 A1.

E. A. Friis, R. S. Lakes, J. B. Park: Negative Poisson's ratio polymeric and metallic foams, Journal of Materials Science, 23, 1998, 4406-4414 discloses an auxetic material made from a highly ductile copper foam. The auxetic properties are also given to the copper foam by a suitable plastic deformation.

Only thermoplastic polymers and highly ductile metals are suitable for making the known auxetic materials by plastic deformation of foams. The three-dimensional structures thereby created by chance are not periodic and only partially have an auxetic structure. The auxetic properties of these materials cannot be adjusted precisely.

The object of the invention is to eliminate the disadvantages from prior art. In particular, an auxetic material is to be specified which can be made from a plurality of different materials. According to a further goal of the invention, the auxetic properties should also be adjustable.

This object is solved by the features of claims 1 and 16 to 18. Useful embodiments of the invention result from the features of claims 2 to 15.

According to the provisions of the invention, an auxetic material is suggested which is created from a periodic arrangement of three-dimensional structural elements, wherein each of the structural elements comprises a first and at least three second supporting elements, wherein the first and the second supporting elements are connected at a common node with their one ends, and wherein a first angle between the first supporting element and the second supporting elements is less than 90°.—The suggested material comprises a structural framework which has auxetic properties due to its special design of the three-dimensional structural elements constituting it. The structural framework results from a periodic arrangement of the structural elements which are connected with one another. At a structural plane, the periodicity is usefully equal to 1. In other words, each structural element is directly connected with a further structural element. The periodicity can exist in three linearly independent spacial directions. The free ends of the supporting elements are usefully connected with one another. The structural elements are connected with one another in such a manner that their nodes and supporting elements do not touch each other during a deformation of the lattice. The term “deformation” is understood to mean a reversible distortion of the lattice.

The structural elements can be varied within the framework of this invention; for example, by changing the number of the second supporting elements and/or the first angle and/or a length of the first and/or second supporting elements. The first and/or second supporting elements can be designed straight, curved or wavy. By changing the structural element, it is possible to adjust desired auxetic properties. The structural elements can be made of any suitable material, in particular also ceramics, all metals or even polymers. The class of auxetic materials can be expanded significantly by this. Totally new possibilities for adjusting material and component properties result particularly also from the combination of auxetic materials and non-auxetic materials.

With regard to the design of the three-dimensional structural element, it has been shown to be advantageous that a second angle between each two adjacent second supporting elements is of the same size. In other words, all second angles have the same size. Furthermore, the second supporting elements can have the same length. A length of the first supporting element can differ from the length of the second supporting element. However, it can also be that the first supporting element and the second supporting elements have the same length.

Advantageously, the structural elements connected with each other create a structural layer for which the nodes are located at one structural plane and the first supporting elements extend vertically from the structural plane in the same direction. In the structural layer, the structural elements are thus connected with each other by the ends of the second supporting elements. The connection of three structural elements with their second supporting elements results in a structural layer with a honeycomb-like structure.

A three-dimensional structural framework created by the structural elements is created by structural layers arranged one on top of the other. The structural layers can be usefully arranged one on top of the other in such a manner that their structural planes run essentially parallel. In this case, the structural layers are supported one on top of the other by the first supporting elements.

According to a further embodiment, the structural layers are arranged periodically in stacks of twos or threes one on top of the other. Here a periodicity in a z direction is thus preferably equal to 2 or 3. The different periodic arrangement of the structural layers can be used to make structural frameworks with different properties.

The structural layers are advantageously connected with each other via the first supporting elements. The connection points created for the connection of the structural layers can be located in a connection point plane which is essentially parallel to the structural plane. At least three second supporting elements of a structural layer as well as a first supporting element of a further structural layer arranged on top are advantageously connected with each other at a connection point.

The structural elements can be made of metal, preferably of titanium, a titanium, cobalt chromium or nickel-base alloy, steel, magnesium, shape memory alloys, in particular NiTi. Likewise, it is possible to make the structural elements from plastic, preferably polyamide, polyetheretherketone, or similar. Moreover, it is possible to make the structural elements from ceramics, preferably SiC, Al₂O₃, hydroxylapatite or similar. According to a useful embodiment, the structural elements are coated with a coating material. This can be, for example, hydroxylapatite, tantalum, TiN, TiC or diamond. It can also be that the surface of the structural elements is modified, for example by etching or similar.

The suggested auxetic material can be used in many areas. It has been shown to be particularly useful to use the auxetic material as a bone substitute substance or as part of a bone substitute substance or implant. To this extent, it is expected that due to the auxetic properties during increase and reduction stress, a pump effect will result which supports the supply of the biological tissue. In particular, the auxetic material can also be made from a reabsorbable material such as magnesium, hydroxylapatite. The suggested auxetic material is also particularly suitable for the making of intervertebral disk materials, for back lining, for example of a knee joint implant or as a replacement for bone marrow.

Aside from this, the suggested auxetic material can be used to make noise-absorbing materials, and to make materials for protection from an impact or a collision as well as to make adaptive filters with variable pore size.

In addition, the auxetic material can be utilized as a framework to make composite materials, for example by infiltration with polymers, metals or ceramic materials.

Conventional Rapid Manufacturing or additive manufacturing processes, for example selective laser or electron beam casting are suitable for making the suggested auxetic material. But it is also possible to make the suggested auxetic material with a casting process, preferably as investment casting. In addition, it is conceivable to use other suitable manufacturing processes, such as lithography, electroforming, molding as well as micro processing techniques. Conventional processes, such as physical or chemical vapor deposition, galvanic coating processes, powder coating processes and similar are suitable for coating an auxetic structural framework provided by the invention.

Examples will now be used to describe the invention in more detail based on the drawings. The figures are listed below:

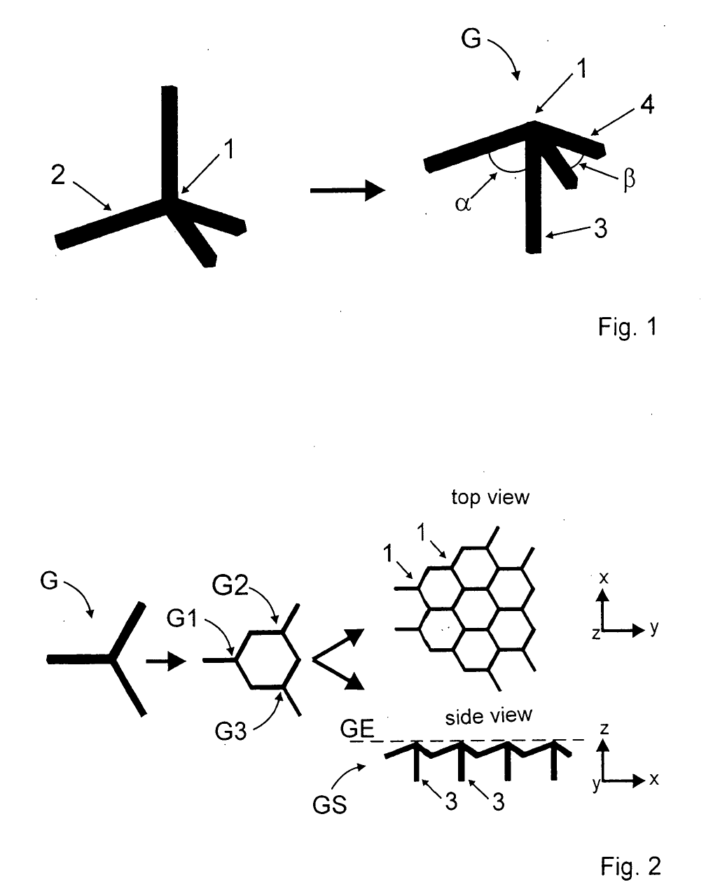

FIG. 1 the derivation of a structural element,

FIG. 2 the creation of a structural layer from the structural element as per FIG. 1,

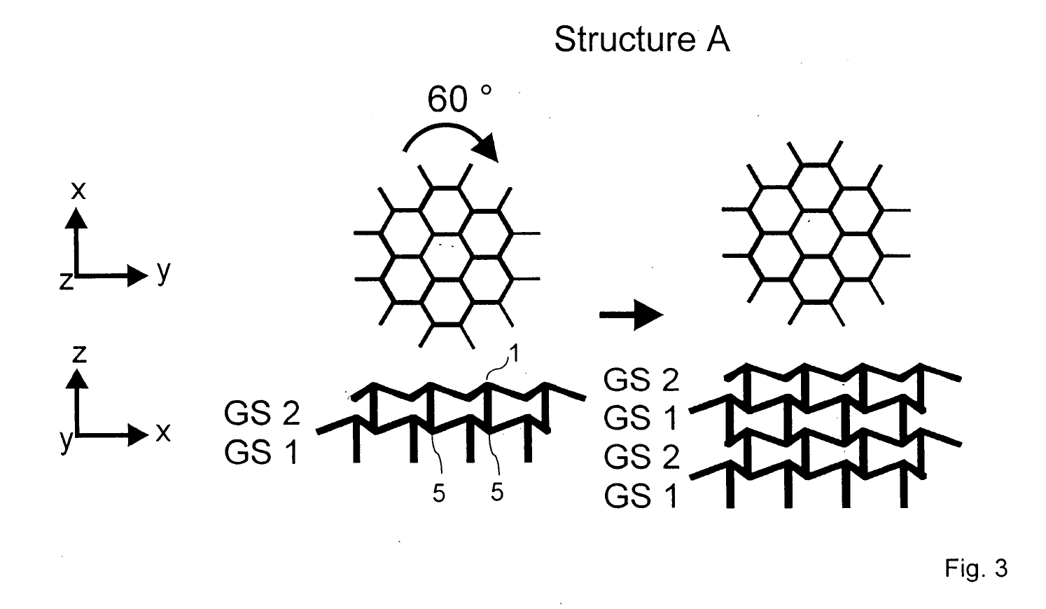

FIG. 3 a first structural framework using the structural layer as per FIG. 2 and

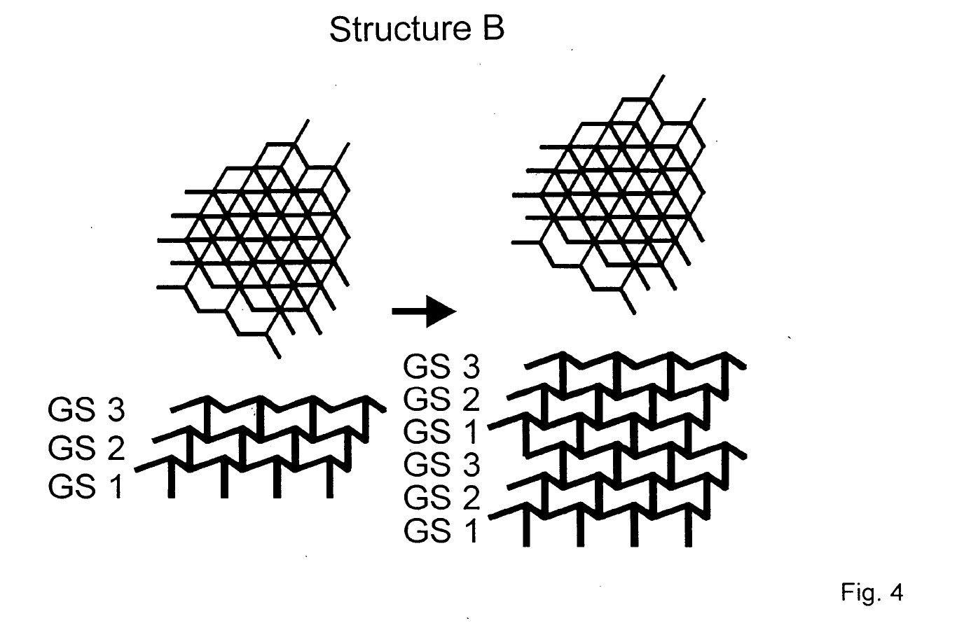

FIG. 4 a second structural framework using the structural layer as per FIG. 2.

The left-hand view of FIG. 1 shows a tetrahedral structure as it is implemented in the diamond lattice, for example. Four arms 2 of the same length extend from a node 1 with the known tetrahedron angle of 109.5°. A structural element G provided by the invention can be derived from such a tetrahedral structure by mirroring three arms on a symmetry plane running perpendicularly to the fourth arm and through the node 1. Such a structural element G is shown in the right-hand view of FIG. 1. It is created from a first supporting element 3 and three second supporting elements 4. The first 3 and the second supporting elements 4 are connected by the node 1. The supporting elements are preferably shaped like rods or poles. They usefully have a circular-shaped cross section. A first angle α between the first 3 and each of the second supporting elements 4 is identical. The angle α is less than 90°. It is usefully located in the area from 85 to 30°, preferably in the area from 85 to 60° or from 85 to 70°. A second angle β between two adjacent supporting elements 4 is also identical. It is 109.5° for the example shown in FIG. 1. The size of the second angle β depends on the size of the first angle α. In the structural element G shown in FIG. 1, the first 3 and the second supporting elements 4 have the same length. However, it is also conceivable that the first supporting element 3 is longer or shorter than the second supporting elements 4. Moreover, it is conceivable that the second supporting elements 4 also have different lengths. In this case, it may be necessary to deviate from angle equality between the second supporting elements 4. In other words, there can also be second angles β of different sizes between the second supporting elements 4.

FIG. 2 shows the formation of a structural layer GS using the structural element G shown in FIG. 1. Three structural elements G1, G2, G3 are connected with each other with the free ends of two second supporting elements 4 each in such a manner that a honeycomb-like structure is created in the projection onto the xy-plane. The first supporting elements 3 are perpendicular to a structural plane GE created by the nodes 1.

FIG. 3 shows the formation of a first structural framework A by stacking several of the structural layers GS shown in FIG. 2 one on top of the other. A second structural layer GS2 is supported in connection points 5 by its first supporting elements 3 on a first structural layer GS1. At each of the connection points 5, at least three second supporting elements 4 of the first structural layer GS1 as well as a first supporting element 3 of the second structural layer GS2 located above are connected with each other. The connection points 5 form a connection point plane VE which runs approximately parallel to the structural plane GE. The second structural layer GS2 is rotated by 60° in comparison to the first structural layer GS1, wherein the axis of rotation is perpendicular to the structural layer GS. The structural layer sequence of first structural layer GS1 and second structural layer GS2 is periodically stacked one on top of the other to establish the first structural framework A. The first structural framework A as shown in the right-hand view of FIG. 3 results.

In the second structural framework B shown in FIG. 4, a structural layer sequence consists of a first structural layer GS1, a second structural layer GS2 and a third structural layer GS3 which are arranged without rotation, respectively, but with a translation in the structural plane GE in the direction of the projection of a second structural element 4 onto the structural plane GE. The second structural framework B is created from a periodic stacking on top of one another of the structural layer sequence created from the three structural layers GS1, GS2 and GS3.

The suggested structural frameworks A, B can be made from a plurality of different materials, for example, with Rapid Prototyping process, casting process or similar. By varying the geometry, in particular the length or the width of the supporting elements 3, 4 as well as the angles α, β provided between the supporting elements 3, 4, the auxetic and also other properties of the suggested material can be adjusted.

Auxetic structural frameworks can also be made by using other structural elements G. Suitable structural elements G can also have four second supporting elements 4 and a first supporting element 3, for example.

The suggested auxetic material is particularly suitable for making bone substitute materials. A length of the supporting elements 3, 4 is preferably 0.5 to 3 mm. The diameter of the supporting elements 3, 4 which are preferably round in the cross section is between 0.1 and 1 mm and can be adjust variably in the structure.

### REFERENCE SIGNS

1 node
2 arm
3 first supporting element
4 second supporting element
5 connection point
α first angle
β second angle
A, B structural framework
G, G1, G2, G3 structural element
GE structural plane
GS, GS1, GS2, GS3 structural layer
VE connection point plane

1. Auxetic material composed of a periodical arrangement of three-dimensional structural elements (G, G1, G2, G3) connected to each other, wherein each of the structural elements (G, G1, G2, G3) comprises a first (3) and at least three second supporting elements (4), wherein the first (3) and the second supporting elements (4) are connected at a common node (1) with their one ends, and wherein a first angle (α) between the first supporting element (3) and the second supporting elements (4) is less than 90°.

2. Auxetic material as defined in claim 1, wherein a second angle (β) between two adjacent second supporting elements (4) is respectively of the same size.

3. Auxetic material as defined in claim 1, wherein the second supporting elements (4) have the same length.

4. Auxetic material as defined in claim 1, wherein the first supporting element (3) and the second supporting elements (4) have the same length.

5. Auxetic material as defined in claim 1, wherein the structural elements (G, G1, G2, G3) connected to each other form a structural layer (GS, GS1, GS2, GS3) in which the nodes (1) are located in a structural plane (GE) and the first supporting elements (3) extend vertically in the same direction from the structural plane (GE).

6. Auxetic material as defined in claim 1, wherein a three-dimensional structural framework (A, B) created by the structural elements (G, G1, G2, G3) is created by structural layers (GS, GS1, GS2, GS3) which are arranged one on top of the other.

7. Auxetic material as defined in claim 1, wherein the structural layers (GS, GS1, GS2, GS3) are arranged one on top of the other in such a manner that their structural planes (GE) run essentially parallel.

8. Auxetic material as defined in claim 1, wherein the structural layers (GS, GS1, GS2, GS3) are arranged periodically in stacks of 2 or 3 one on top of the other.

9. Auxetic material as defined in claim 1, wherein the structural layers (GS, GS1, GS2, GS3) are connected with one another via the first supporting elements (3).

10. Auxetic material as defined in claim 1, wherein the connection points (3) formed to connect the structural layers (GS, GS1, GS2, GS3) are located in a connection point plane (VE) which is essentially parallel to the structural plane (GE).

11. Auxetic material as defined in claim 1, wherein at least three second supporting elements (4) of a structural layer (GS, GS1, GS2, GS3) as well as a first supporting element (3) of a further structural layer (GS, GS1, GS2, GS3) located on top are connected with one another at a connection point (5).

12. Auxetic material as defined in claim 1, wherein the structural elements (G, G1, G2, G3) are made of metal, preferably titanium, a titanium or cobalt chromium or nickel base alloy, Mg, steel, shape memory alloys, in particular NiTi.

13. Auxetic material as defined in claim 1, wherein the structural elements (G, G1, G2, G3) are made of plastic, preferably polyamide, polyethereketone.

14. Auxetic material as defined in claim 1, wherein the structural elements (G, G1, G2, G3) are made of ceramics, preferably SiC, Al₂O₃, hydroxylapatite.

15. Auxetic material as defined in claim 1, wherein the structural elements (G, G1, G2, G3) are coated with a coating material, preferably hydroxylapatite, tantalum, TiN, TiC or diamond.

16. Bone substitute materials, comprising the auxetic material as defined in claim 1.

17. Implant, comprising the auxetic material as defined in claim 1.

18. Composite material, comprising the auxetic material as defined in claim 1.
